(19) United States
(12) Patent Application Publication
(10) Pub. No.: US 2022/0211780 A1
(43) Pub. Date: Jul. 7, 2022

(54) Methods And Compositions For Treating Liver Disorders

(71) Applicant: Pendulum Therapeutics, Inc., San Francisco, CA (US)

(72) Inventors: Orville Kolterman, La Jolla, CA (US); Fanny Perraudeau, San Francisco, CA (US); James Bullard, San Francisco, CA (US); John Eid, San Francisco, CA (US); Colleen Cutcliffe, Menlo Park, CA (US)

(21) Appl. No.: 17/610,398

(22) PCT Filed: May 20, 2020
(86) PCT No.: PCT/US2020/033870

§ 371 Date: Nov. 10, 2021

Related U.S. Application Data
(60) Provisional application No. 62/850,773, filed on May 21, 2019.

(51) Int. Cl. A61K 35/747; A61K 35/745; A61K 35/742; A61K 35/741; A61K 35/744; A61P 1/16; A61K 9/48

(52) U.S. Cl. CPC A61K 35/747; A61K 35/745; A61K 35/742; A61K 9/4891; A61K 35/744; A61P 1/16; A61K 35/741

(57) ABSTRACT

Compositions and methods are provided for treating, mitigating, managing, reducing or preventing the onset of symptoms, signs or indicators of liver disorders as well as the disorders themselves. The compositions and methods include microbial compositions that are selected to improve gut function in the subjects to which they are administered, so as to bring about treatment of liver disorders and/or the signs, symptoms and indicators of those disorders.

### CROSS REFERENCE

This application claims priority to U.S. Provisional Patent Application No. 62/850,773, filed May 21, 2019, which is incorporated herein by reference in its entirety.

### BACKGROUND

It can be stated, and likely with little argument, that the most significant inputs into human viability, health and wellness, barring physical injury, come from three areas: (1) the structural make-up of the human body, as templated by the human genome, and as built through its replication, expression and transcription into the biological structures that make up human tissue; (2) the inputs to that human structure, both in terms of environmental inputs as well as ingested inputs, e.g., foods; and (3) the processes or interfaces by which the human structure processes those inputs, including both integral physiological properties of tissues to process those inputs, as well as the symbiotic processes involving the totality of microbiota that are upon and within the human structure, and which serve as intermediaries and/or interfaces in the processing of those inputs for transfer to, or the protection of, the physiological structures of the body.

A great deal of research has gone into examining the genetic and physical aspects of human health and wellness, and many discoveries have resulted that have provided significant health benefits, in terms of diagnosing and treating a wide variety of health disorders, as well as advising habits of healthy living. Likewise, the impacts of environmental and nutritional inputs on human health have been researched at great length, resulting in a greater understanding of how our environments and diets influence our health and well-being.

While researchers have continued to advance understandings of human health and disease through advances in technologies for analysis of living systems, many aspects of health and disease still remain an enigma, resulting in an inability to identify causes and/or treatments of a large number of human diseases. This inability is particularly manifested in a large number of complex diseases that have gained prevalence over the past century.

By way of example, for complex degenerative diseases like Alzheimer's Disease, Parkinson's Disease, amyotrophic lateral sclerosis (ALS), autism, and many other neurological disorders that have seen a rise in occurrence, researchers have tried and failed to definitively identify underlying genetic or other causes. As a result, attempts at identifying potential treatments for these diseases have regularly failed. Similarly, the causes and potential treatment of a large class of metabolic disorders has similarly defied efforts at finding their root causes, and thus their potential treatment, despite early beliefs in genetic or nutritional root causes. These include disorders such as diabetes mellitus (type 1 and type 2), dyslipidemia, insulin resistance, inflammatory bowel disease, irritable bowel syndrome, obesity, and associated liver diseases, such as non-alcoholic fatty liver disease (NAFLD), including the progression from non-alcoholic steatohepatitis (NASH), through liver fibrosis and cirrhosis.

In the absence of a clear nexus between many of these metabolic disorders and either an underlying genetic or environmental cause, researchers have begun exploring the role of the gut microbiome in the etiology of these disorders. While this research has provided tantalizing clues as to a microbiome component in the progression of these disorders, to date, there has not been identified any key component of microbiome function that is present or lacking in this disease progression, and moreover, any potential strategy for remedying or mitigating that progression. The present disclosure addresses these and many other needs.

### SUMMARY

Compositions and methods are provided for treating, mitigating, managing, reducing or preventing the onset of symptoms, signs or indicators of liver disorders as well as the disorders themselves. The compositions and methods include microbial compositions that are selected to improve gut function in the subjects to which they are administered, so as to bring about treatment of liver disorders and/or the signs, symptoms and indicators of those disorders.

In some aspects, the disclosure provides a method of treating a liver disorder in a subject in need thereof, comprising administering to the subject an effective amount of a composition comprising a consortium of isolated and purified viable microbial populations to reduce a serum level of one or more of aspartate transaminase (AST) and alanyl transaminase (ALT) enzymes by at least 5 IU/L in the subject as compared to ALT and/or AST levels in the subject prior to administering the consortium of isolated and purified microbial species.

In some embodiments, the liver disorder is selected from the group consisting of nonalcoholic steatohepatitis (NASH), non-alcoholic fatty liver disease (NAFLD), liver fibrosis, cirrhosis, alcohol induced liver disease, and drug induced liver injury. In some embodiments, the liver disorder is selected from the group consisting of: NASH and NAFLD. In some embodiments, the liver disorder is concurrent with a metabolic disorder. In some embodiments, the metabolic disorder is selected from the group consisting of type 1 diabetes mellitus, type 2 diabetes mellitus, insulin resistance, and obesity. In some embodiments, the method comprises administering to the subject at least 1×10\hat{ }8 CFUs of the microbial populations per day. In some embodiments, the method comprises administering to the subject at least 1×10\hat{ }9 CFUs of the microbial populations per day. In some embodiments, the method comprises administering to the subject at least 1×10\hat{ }10 CFUs of the microbial populations per day. In some embodiments, the method comprises administering to the subject at least 1×10\hat{ }8 CFUs of the microbial populations at least two times per day. In some embodiments, the method comprises administering to the subject at least 1×10\hat{ }8 CFUs of the microbial populations at least three times per day. In some embodiments, the method comprises administering to the subject at least 1×10\hat{ }8 CFUs of the microbial populations at least four times per day. In some embodiments, the administering is continued for at least one week. In some embodiments, the administering is continued for at least two weeks. In some embodiments, the administering is continued for at least four weeks. In some embodiments, the administering is continued for at least six weeks. In some embodiments, the administering is continued for at least eight weeks. In some embodiments, the administering is continued for at least twelve weeks. In some embodiments, the administering is continued for at least eighteen weeks. In some embodiments, the administering is continued for at least twenty-six weeks. In some embodiments, the administering is continued for at least one year. In some embodiments, the microbial populations are formulated in an ingestible form and are administered orally. In some embodiments, the ingestible form comprises a pill. In some embodiments, the ingestible form comprises a capsule. In some embodiments, the ingestible form is a bar. In some embodiments, the ingestible form comprises a chewable tablet or gummy. In some embodiments, the ingestible form comprises a powder. In some embodiments, the microbial species are microencapsulated in the ingestible form. In some embodiments, the consortium comprises 2 or more microbial populations selected from primary fermenters and secondary fermenters. In some embodiments, the consortium comprises 2 or more microbial populations selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof. In some embodiments, the consortium comprises 2 or more microbial populations selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and Faecalibacterium prausnitzii. In some embodiments, the administration reduces the serum level of one or more of aspartate transaminase (AST) and alanyl transaminase (ALT) enzymes by at least 10 IU/L in the subject as compared to ALT and/or AST levels in the subject prior to administering the consortium of isolated and purified microbial species. In some embodiments, the administration reduces the serum level of one or more of aspartate transaminase (AST) and alanyl transaminase (ALT) enzymes by at least 20 IU/L in the subject as compared to ALT and/or AST levels in the subject prior to administering the consortium of isolated and purified microbial species. In some embodiments, the administration reduces the serum level of one or more of aspartate transaminase (AST) and alanyl transaminase (ALT) enzymes by at least 50 IU/L in the subject as compared to ALT and/or AST levels in the subject prior to administering the consortium of isolated and purified microbial species. In some embodiments, the administration reduces the serum level of one or more of aspartate transaminase (AST) and alanyl transaminase (ALT) enzymes by at least 100 IU/L in the subject as compared to ALT and/or AST levels in the subject prior to administering the consortium of isolated and purified microbial species.

In some aspects, the disclosure provides a method of reducing one or more elevated indicators of liver injury or disease in a subject, comprising administering to a subject having one or more elevated indicators of liver injury an effective amount of a composition comprising one or more purified viable microbial populations, wherein the one or more purified viable microbial populations are capable of producing butyrate in a gut of the subject, such effective amount resulting in a reduction on the one or more indicators of liver disease in the subject.

In some embodiments, the one or more indicators of liver injury are selected from the group consisting of: AST, ALT, AST:ALT ratio, fibrosis score (“NFS”), the FIB-4 index, the aspartate aminotransferase (“AST”) platelet ratio index (“APRI”), enhanced liver fibrosis (“ELF”) panels, transient elastography (“TE”), magnetic resonance (“MR”) elastography, acoustic radiation force impulse imaging, and supersonic shear wave elastography.

In some aspects, the disclosure provides a method of lowering one or more of ALT and AST serum levels in a subject suffering from a liver disorder or at risk of suffering from a liver disorder, comprising administering to the subject an effective amount of a consortium of isolated and purified microbial species to lower the one or more ALT and AST serum levels in the subject.

In some embodiments, the subject has type 2 diabetes. In some embodiments, the subject has been diagnosed with a liver disorder. In some embodiments, the liver disorder is NAFLD, NASH, liver fibrosis, cirrhosis, or DILI. In some embodiments, the subject is being concurrently administered a drug with known liver toxicity.

In some aspects, the disclosure provides a method of reducing liver toxicity of one or more drug compounds known to have liver toxicity, comprising: co-administering with the one or more drug compounds, a composition comprising an effective amount a consortium of isolated and purified microbial species in an effective amount to lower one or more indicators of liver injury.

In some aspects, the disclosure provides a method of treating a liver disorder in a subject in need thereof, comprising: administering to the subject an effective amount of a consortium of isolated and purified microbial species to mitigate the liver disorder.

### BRIEF DESCRIPTION OF THE FIGURES

FIG. 1 illustrates an example of a human digestive pathway, and gut microbiome mediated production of butyrate therein.

FIG. 2 illustrates clinical reductions for in liver enzyme biomarkers of liver disease in subjects following treatment with microbial populations as described herein.

### DETAILED DESCRIPTION

### I. General

Microbiome interventions have previously been described for use in treating metabolic disorders like type-2 diabetes, obesity and related diseases. In particular, oral administration of compositions that include commensal microbial populations have been shown to significantly reduce post prandial glucose levels and HbA1c levels in type-2 diabetics (See co-pending U.S. patent application Ser. No. 62/801,983, filed Feb. 6, 2019, and co-pending PCT Application No. PCT/US19/52694, filed Sep. 24, 2019, each of which is incorporated herein by reference in its entirety for all purposes). As described herein, however, administration of microbial compositions as a microbiome intervention, may also provide a method for treatment or mitigation of additional disorders, such as hepatic disorders associated with drug or other toxicity, and/or those associated with metabolic disorders, such as NAFLD and NASH, as well as other liver disorders.

In some cases, administration of the compositions described herein may result in a treatment or mitigation of liver associated disorders such as NASH, NAFLD, and progressions of such disorders, such as liver fibrosis and cirrhosis. In certain cases, these disorders or the increased risk of such disorders are in patients or subjects where they are associated or concurrent with other metabolic disorders in such patients, such as type 1 diabetes, type 2 diabetes, insulin resistance, obesity, or the like, and as such, the treatment methods may be applied to these patient groups in treatment, or delaying onset, as the case may be, of liver disorders associated with these conditions. In other cases, administration of the compositions described herein may result in the treatment or mitigation of other hepatic disorders, such as liver injury associated with drug toxicity, excessive alcohol consumption, or the like. For ease of discussion, the above described liver disorders and/or injuries are collectively referred to herein as liver disorders.

### II. Compositions

The compositions described herein include microbial compositions that may be used to treat or otherwise mitigate the symptoms of liver disorders. In particular, in some cases are provided methods of mitigating symptoms, treating or managing liver disorders by administering to a subject suffering from such disorder an effective amount of a microbial composition (as further described herein) to affect such mitigation, treatment or management.

These microbial compositions may include naturally occurring microbial strains that may be underrepresented or insufficiently represented in subjects who are suffering from such liver disorders. In some cases, the microbial strains may be under represented in the gut of a subject suffering from a liver disorder or injury relative to their level of representation in the gut of a healthy subject, and thus administration of the microbial compositions may be aimed at restoring a healthy level of such microbes in the gut in order to mitigate symptoms of, treat or manage liver disorders. In other cases, it may be desirable to increase representation of the microbial species in the gut of a subject suffering from such a disorder or injury over levels typically found in the gut of healthy subjects, and thus administration of the microbial compositions may be aimed at achieving such over-representation in order to mitigate symptoms or otherwise treat or manage such liver disorders.

The microbial compositions may include any of a number of different microbial populations. As used herein, a microbial population typically refers to a microbial population that is substantially comprised of a single strain, species or genus, as may be the case when the population is cultured from an isolated and purified subpopulation of such strain, species or genus. Thus, with respect to a given microbial population, such population will be referred to herein as purified or substantially pure if cultured from an isolated microbial species or strain. The resulting population may generally be at least 80% pure as to the stated microbial species or strain, at least 90% pure with respect to other microbial species or strains within that particular population, at least 95% pure, at least 98% pure, at least 99% pure, at least 99.5% pure, or at least 99.9% pure. Conversely, the level of non-desired strains in any particular desired microbial population will be less 20%, less than 10%, less than 5%, less than 2%, less than 1%, less than 0.5% or less than 0.1%. In the case of compositions that comprise a consortium of multiple microbial populations, each population may have the purity described above, either prior to its incorporation into the composition, or, when measured in aggregate as to the consortium. For example, the level of impurities, e.g., other non-desired microbial strains or species, in a consortium of purified populations may be, on a pro rata basis, at or below the levels stated above for each desired population.

Without being bound to any particular theory of operation, it is believed that the microbial compositions described herein play an important role in the metabolism of dietary carbohydrates and energy generation in the human gut. As a result, the enhancement of the populations of these organisms in the gut has been shown to improve symptoms of metabolic disorders, such as type 2 diabetes. Surprisingly, as described elsewhere herein, such enhancements have also demonstrated an ability to improve symptoms and indications of other disorders that may be, in some cases, associated with these metabolic disorders, such as liver disorders or injuries. In particular, microbes involved in the production and absorption of short chain fatty acids are believed to be particularly useful in metabolic processes that can help treat or otherwise mitigate symptoms of liver disease or injury. Examples of these microbes include, for example, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof.

In some cases, the microbial populations may be selected to provide enhanced metabolic function within the gut that may contribute to, among other things, mitigation or treatment of symptoms of liver disorders or injury. By way of example, butyrate is an anti-inflammatory factor that can affect gut permeability. Lower levels of certain butyrate producing bacteria (e.g. Clostridium clusters XIVa and IV) as well as reduced levels of lactate producing bacteria (e.g. Bifidobacterium adolescentis) have been correlated to certain metabolic disorders, such as type II diabetes mellitus (T2D), obesity, and other similar metabolic disorders. There has been shown a strong correlation between subjects having metabolic disorders and the occurrence of liver disorders (see, e.g., Chalassani, et al., Hepatology Vol. 67, No. 1 (2018) 328-357).

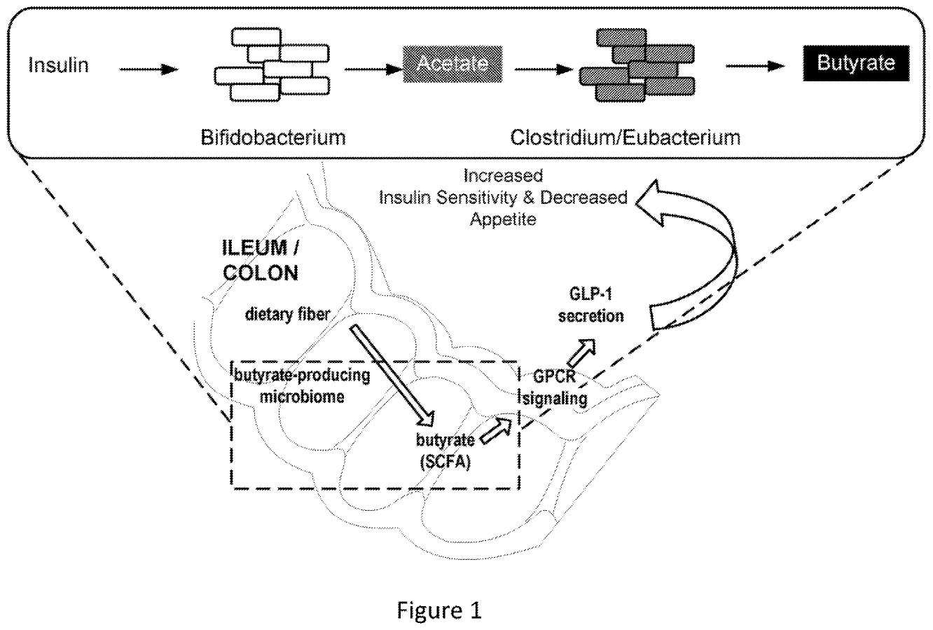

FIG. 1 depicts a digestive pathway that can impact metabolic-related health conditions. Again, without being bound to any particular theory of operation, it is believed that alteration of the pathway using microbial compositions of the invention can correct deficiencies in that pathway in a subject, which, in turn, may lead to mitigation or treatment of liver disorders or injury. As illustrated, in the colon, dietary fiber can be processed by butyrate-producing microorganisms to produce butyrate (i.e. butanoate), which is a short chain fatty acid (SCFA). In turn, butyrate can initiate G-protein coupled receptor (GPCR) signaling, leading to glucagon-like peptide-1 (GLP-1) secretion which can result in increased insulin secretion, increased insulin sensitivity and/or decreased appetite. By altering the butyrate-producing microbiome in a subject, e.g. a subject suffering from T2DM or insulin insensitivity, the pathway can be stimulated. In some patients, insulin secretion may be improved, and in some cases, may be increased and/or restored to pre-diabetic levels with a microbial composition.

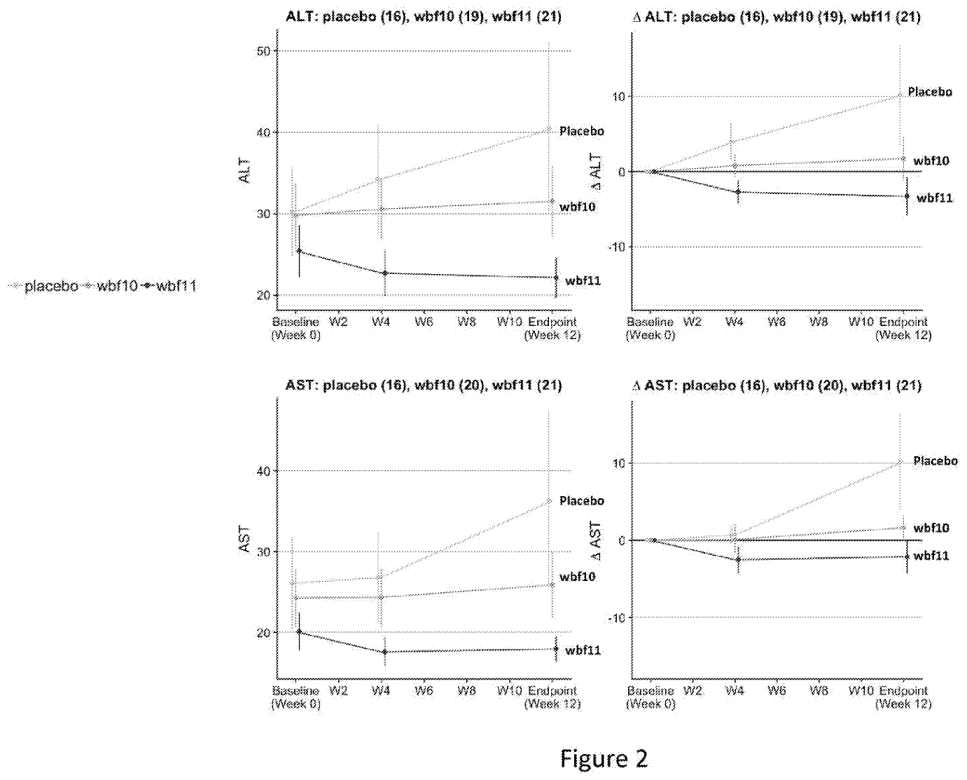

As described herein, clinical trials aimed at determining effects of microbial compositions that include subsets of these microbes in T2D patients (see Provisional U.S. Patent Application No. 62/801,983, previously incorporated herein by reference in its entirety for all purposes) also demonstrated significant improvements in biomarkers associated with liver disorders and liver injury (See Example 1, below, and FIG. 2).

Accordingly, and without being bound to any particular theory of operation, in some aspects of the invention, strains of interest may be chosen in a fashion by identifying a superset of bacteria that play a role in the functional pathway that leads to GLP-1 production (e.g. bacteria that have butyrate kinase, butyrate coenzyme A (CoA), and/or butyrate CoA transferase genes). Butyrate kinase is an enzyme that can belong to a family of transferases, for example those transferring phosphorus-containing groups (e.g., phosphotransferases) with a carboxy group as acceptor. The systematic name of this enzyme class can be ATP:butanoate 1-phosphotransferase. Butyrate kinase can participate in butyrate metabolism. Butyrate kinase can catalyze the following reaction: ADP+butyryl-phosphateATP+butyrate, Butyrate-Coenzyme A, also butyryl-coenzyme A, can be a coenzyme A-activated form of butyric acid. It can be acted upon by butyryl-CoA dehydrogenase and can be an intermediary compound in acetone-butanol-ethanol fermentation. Butyrate-Coenzyme A can be involved in butyrate metabolism.

Butyrate-Coenzyme A transferase, also known as butyrate-acetoacetate CoA-transferase, can belong to a family of transferases, for example, the CoA-transferases. The systematic name of this enzyme class can be butanoyl-CoA:acetoacetate CoA-transferase. Other names in common use can include butyryl coenzyme A-acetoacetate coenzyme A-transferase, and butyryl-CoA-acetoacetate CoA-transferase. Butyrate-Coenzyme A transferase can catalyze the following chemical reaction: butanoyl-CoA+acetoacetatebutanoate+acetoacetyl-CoA

Butyryl-CoA dehydrogenase can belong to the family of oxidoreductases, for example, those acting on the CH—CH group of donor with other acceptors. The systematic name of this enzyme class can be butanoyl-CoA:acceptor 2,3-oxidoreductase. Other names in common use can include butyryl dehydrogenase, unsaturated acyl-CoA reductase, ethylene reductase, enoyl-coenzyme A reductase, unsaturated acyl coenzyme A reductase, butyryl coenzyme A dehydrogenase, short-chain acyl CoA dehydrogenase, short-chain acyl-coenzyme A dehydrogenase, 3-hydroxyacyl CoA reductase, and butanoyl-CoA:(acceptor) 2,3-oxidoreductase. Non-limiting examples of metabolic pathways that butyryl-CoA dehydrogenase can participate in include: fatty acid metabolism; valine, leucine and isoleucine degradation; and butanoate metabolism. Butyryl-CoA dehydrogenase can employ one cofactor, FAD. Butyryl-CoA dehydrogenase can catalyze the following reaction: butyryl-CoA+acceptor2-butenoyl-CoA+reduced acceptor.

Beta-hydroxybutyryl-CoA dehydrogenase or 3-hydroxybutyryl-CoA dehydrogenase can belong to a family of oxidoreductases, for example, those acting on the CH—OH group of donor with NAD+ or NADP+ as acceptor. The systematic name of the enzyme class can be (S)-3-hydroxybutanoyl-CoA:NADP+oxidoreductase. Other names in common use can include beta-hydroxybutyryl coenzyme A dehydrogenase, L(+)-3-hydroxybutyryl-CoA dehydrogenase, BHBD, dehydrogenase, L-3-hydroxybutyryl coenzyme A (nicotinamide adenine, dinucleotide phosphate), L-(+)-3-hydroxybutyryl-CoA dehydrogenase, and 3-hydroxybutyryl-CoA dehydrogenase. Beta-hydroxybutyryl-CoA dehydrogenase enzyme can participate in benzoate degradation via coa ligation. Beta-hydroxybutyryl-CoA dehydrogenase enzyme can participate in butanoate metabolism. Beta-hydroxybutyryl-CoA dehydrogenase can catalyze the following reaction: (S)-3-hydroxybutanoyl-CoA+NADP.sup.+3-acetoacetyl-CoA+NADPH+H.sup.+

Crotonase can comprise enzymes with, for example, dehalogenase, hydratase, isomerase activities. Crotonase can be implicated in carbon-carbon bond formation, cleavage, and hydrolysis of thioesters. Enzymes in the crotonase superfamily can include, for example, enoyl-CoA hydratase which can catalyse the hydration of 2-trans-enoyl-CoA into 3-hydroxyacyl-CoA; 3-2trans-enoyl-CoA isomerase or dodecenoyl-CoA isomerise (e.g., EC 5.3.3.8), which can shift the 3-double bond of the intermediates of unsaturated fatty acid oxidation to the 2-trans position; 3-hydroxbutyryl-CoA dehydratase (e.g., crotonase; EC 4.2.1.55), which can be involved in the butyrate/butanol-producing pathway; 4-Chlorobenzoyl-CoA dehalogenase (e.g., EC 3.8.1.6) which can catalyze the conversion of 4-chlorobenzoate-CoA to 4-hydroxybenzoate-CoA; dienoyl-CoA isomerase, which can catalyze the isomerisation of 3-trans,5-cis-dienoyl-CoA to 2-trans,4-trans-dienoyl-CoA; naphthoate synthase (e.g., MenB, or DHNA synthetase; EC 4.1.3.36), which can be involved in the biosynthesis of menaquinone (e.g., vitamin K2); carnitine racemase (e.g., gene caiD), which can catalyze the reversible conversion of crotonobetaine to L-carnitine in Escherichia coli; Methylmalonyl CoA decarboxylase (e.g., MMCD; EC 4.1.1.41); carboxymethylproline synthase (e.g., CarB), which can be involved in carbapenem biosynthesis; 6-oxo camphor hydrolase, which can catalyze the desymmetrization of bicyclic beta-diketones to optically active keto acids; the alpha subunit of fatty acid oxidation complex, a multi-enzyme complex that can catalyze the last three reactions in the fatty acid beta-oxidation cycle; and AUH protein, which can be a bifunctional RNA-binding homologue of enoyl-CoA hydratase.

Thiolases, also known as acetyl-coenzyme A acetyltransferases (ACAT), can convert two units of acetyl-CoA to acetoacetyl CoA, for example, in the mevalonate pathway. Thiolases can include, for example, degradative thiolases (e.g., EC 2.3.1.16) and biosynthetic thiolases (e.g., EC 2.3.1.9). 3-ketoacyl-CoA thiolase, also called thiolase I, can be involved in degradative pathways such as fatty acid beta-oxidation. Acetoacetyl-CoA thiolase, also called thiolase II, can be specific for the thiolysis of acetoacetyl-CoA and can be involved in biosynthetic pathways such as poly beta-hydroxybutyric acid synthesis or steroid biogenesis.

As shown in FIG. 1, production of butyrate can involve two major phases or microbes, for example, a primary fermenter and a secondary fermenter. The primary fermenter can produce intermediate molecules (e.g. lactate, acetate) when given an energy source (e.g. fiber). The secondary fermenter can convert the intermediate molecules produced by the primary fermenter into butyrate. Many of these primary and secondary fermenters will express enzymes involved in this butyrate pathway, such as the following non-limiting enzyme examples: butyryl-CoA dehydrogenase, beta-hydroxybutyryl-CoA dehydrogenase or 3-hydroxybutyryl-CoA dehydrogenase, crotonase, electron transfer protein a, electron transfer protein b, and thiolase.

Non-limiting examples of primary fermenters may include such microbes as Akkermansia muciniphila, Bifidobacterium adolescentis, Bifidobacterium infantis and Bifidobacterium longum. Non-limiting examples of secondary fermenters may include such microbes as Clostridium beijerinckii, Clostridium butyricum, Clostridium indolis, Eubacterium hallii, and Faecalibacterium prausnitzii.

With reference to these exemplary microbial species, Akkermansia muciniphila is a gram negative, strict anaerobe that can play a role in mucin degradation. Levels of Akkermansia muciniphila can be reduced in subjects with metabolic disorders, for example, obesity and T2DM. Akkermansia muciniphila may protect against metabolic disorders, for example, through increased levels of endocannabinoids that control inflammation, the gut barrier, and gut peptide secretion. Akkermansia muciniphila can serve as a primary fermenter, and in some cases, be combined with any one or more of the secondary fermenters described herein. Bifidobacterium adolescentis can be a gram-positive anaerobe, which can be found in healthy human gut from infancy. Bifidobacterium adolescentis can synthesize B vitamins Bifidobacterium adolescentis can serve as a primary fermenter, and in some cases, be combined with any one or more of the secondary fermenters described herein. Bifidobacterium infantis can be a gram-positive, catalase negative, micro-aerotolerant anaerobe. Bifidobacterium infantis can serve as a primary fermenter, and in some cases, be combined with any one or more of the secondary fermenters described herein. Bifidobacterium longum can be a gram-positive, catalase negative, micro-aerotolerant anaerobe. Bifidobacterium longum can serve as a primary fermenter, and in some cases, be combined with any one or more of the secondary fermenters described herein. Clostridium beijerinckii can be a gram-positive, strict anaerobe that belongs to Clostridial cluster I. Clostridium beijerinckii can serve as a secondary fermenter, and in some cases, be combined with any one or more of the primary fermenters described herein. Clostridium butyricum can be a gram-positive, strict anaerobe that can serve as a secondary fermenter, and in some cases, be combined with any one or more of the primary fermenters described herein. Clostridium indolis can be a gram-positive, strict anaerobe that belongs to Clostridial cluster XIVA. Clostridium indolis can serve as a secondary fermenter, and in some cases, be combined with any one or more of the primary fermenters described herein. Eubacterium hallii can be a gram-positive, anaerobe that belongs to Arrangement A Clostridial cluster XIVA. Eubacterium hallii can serve as a secondary fermenter, and in some cases, be combined with any one or more of the primary fermenters described herein. Faecalibacterium prausnitzii can be a gram-positive, anaerobe belonging to Clostridial cluster IV. Faecalibacterium prausnitzii can be one of the most common gut bacteria and the largest butyrate producer. Faecalibacterium prausnitzii can serve as a secondary fermenter, and in some cases, be combined with any one or more of the primary fermenters described herein.

In some embodiments, the microbial composition comprises Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, or a combination thereof.

In some embodiments, the microbial composition comprises Akkermansia muciniphila and Eubacterium hallii. In some embodiments, the microbial composition comprises Bifidobacterium infantis, Clostridium beijerinckii, and Clostridium butyricum. In some embodiments, the microbial composition comprises Akkermansia muciniphila, Bifidobacterium infantis, Clostridium beijerinckii, Clostridium butyricum, and Eubacterium hallii.

In some embodiments, the microbial composition comprises Akkermansia muciniphila, Eubacterium hallii and one or more of Bifidobacterium infantis, Clostridium beijerinckii, or Clostridium butyricum.

In some embodiments, the microbial population comprises an rRNA sequence comprising at least 85%, 90%, 91%, 92%, 93%, 94%, 95%, 96%, 97%, 98%, or 99% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

In some embodiments, the composition is substantially animal product-free. In some embodiments, the composition is substantially free of dairy-derived components. In some embodiments, the composition is completely free of any products of animal-origin or any dairy-derived components.

In some embodiments, the microbial composition comprises at least one species that is lyophilized. In some embodiments, the microbial composition comprises at least one species that is non-viable.

A combination of primary and secondary fermenters can be used to produce butyrate in a subject, which, without being bound to any particular theory of operation or mechanism of action, is believed to mitigate metabolic disorders, and, as a result, may treat or otherwise mitigate symptoms of liver disorders. Subsets of a formulation that comprises at least one primary fermenter and at least one secondary fermenter can be used for the treatment and/or mitigate progression of a metabolic health condition, including liver disorders or liver injury. The formulation can additionally comprise a prebiotic.

Accordingly, in some cases, the compositions described herein may include one or more isolated and purified microbial populations. In some cases, a composition may include two or more isolated and purified microbial populations. In other cases, three or more isolated and purified microbial populations may be present within the compositions described herein. In still other cases, 4 or more isolated and purified microbial populations, 5 or more isolated and purified microbial populations, or 6 or more isolated and isolated and purified microbial populations may be included within the compositions.

In some cases, the compositions may comprise at least one primary fermenter and at least one secondary fermenter among the microbial populations present. In some cases, the compositions may include at least one primary fermenter that is selected from the group of Akkermansia muciniphila, Bifidobacterium adolescentis, Bifidobacterium infantis and Bifidobacterium longum. Likewise, in some cases, the compositions may comprise at least one secondary fermenter selected from the group of Clostridium beijerinckii, Clostridium butyricum, Clostridium indolis, Eubacterium hallii, and Faecalibacterium prausnitzii. In some embodiments, a therapeutic composition comprises at least one primary fermenter, at least one secondary fermenter, and at least one prebiotic.

In some cases, the compositions may comprise a mucin degrading or regulating microbe. Examples of mucin degrading or regulating microbes include, for example, Akkermansia muciniphila, Bacterioides fragilis, Bacterioides thetaiotaomicron, Bacterioides vulgatus, Bifidobacterium sp., such as Bifidobacterium bifidum, and others.

The compositions may in some cases comprise a consortium of microbes that include at least 2 different microbial populations within the composition. In other cases, the compositions may comprise at least 3 different microbial populations, at least 4 different microbial populations, at least 5 different microbial populations, at least 6 different microbial populations, and in some cases more than 6 different microbial populations.

### III. Methods of Treatment

Provided herein are methods of treating one or more of a variety of liver disorders. As used herein, the methods described herein may be used to treat subjects who are suffering from one or more liver disorders or liver injuries in order to reduce, remediate, mitigate or slow the progression of such injuries or disorders and/or the symptoms, signs and/or indicators of such disorders in those subjects suffering from these disorders. Additionally or alternatively, the methods described herein may be used to treat subjects who may be at a higher risk for developing these injuries or disorders, and/or the symptoms thereof, in order to prevent or delay onset of such injuries or disorders, and/or the signs or symptoms thereof. For ease of discussion, these interventions (treatment, mitigation, alleviation, prevention, management, remediation, etc.) are referred to collectively as “treat, “treating” and/or “treatment”.

As described herein, the above noted methods may comprise the use of the compositions described herein in the treatment of one or more different liver disorders and/or the signs, symptoms and/or indicators thereof, and/or in the prevention or delay. In particular, in some cases, the methods described herein may be used to treat subjects who are suffering from, or at risk of suffering from such liver associated disorders (or symptoms) and injuries, as non-alcoholic steatohepatitis (NASH), non-alcoholic fatty liver disease (NAFLD), liver fibrosis, cirrhosis, drug induced liver injury (DILI), alcohol induced liver disease, e.g., alcoholic hepatitis, and the like, in order to reduce and/or delay the onset of the signs and/or symptoms of these disorders.

Treatment of these liver disorders typically involves the administration of effective amounts of a composition that comprises the microbial compositions described herein to a subject who is in need of such treatment, including subjects who are suffering from such disorders and subjects who may be in need of prevention or mitigation of the onset of such disorders. In particular, methods of treatment described herein may be intended to be therapeutic, e.g., for the treatment, mitigation or management of liver disorders that have already manifested and/or been diagnosed within a patient or subject.

In other cases, the methods may be prophylactic, e.g., used to treat subjects who may be at increased risk for liver disorders, but who may not yet have manifested the signs or symptoms of the disorder, e.g., subjects who may not yet show overt signs of liver disease, but who are otherwise at elevated risk for such diseases. Examples of such subjects include, e.g., subjects suffering from other metabolic disorders such as diabetes (type 1 and/or type 2), obesity, insulin resistance, and the like. Likewise, subjects at increased risk for liver injury or liver disorders may include subjects being treated with drugs with known or expected liver toxicity issues, or subjects who are otherwise exposed to environments and/or substances that have known liver toxicity issues, e.g., alcoholic subjects, or the like.

In some cases, prophylactic treatment may include co-administration with other therapeutic agents that have heightened risk for liver toxicity. As an example, in some cases, the microbial compositions described herein may be administered prophylactically in conjunction with other medications that are known or suspected of having liver toxicity issues, in order to prevent liver injury or reduce the onset of and/or symptoms of liver toxicity, such as that caused by the co-administered drugs. For example, a number of approved drugs, including over the counter drugs, like acetaminophen, may have the potential to cause liver injury, either when taken in accordance with approved dosing, or when administered at dosages higher than recommended. By co-administering these drugs with the compositions described herein, one may mitigate the toxicity impacts on the liver, thus allowing continued administration of the particular drug, or even elevated dosage of such drugs.

Moreover, in many cases, potential drug candidates may not be approved, or may be abandoned during clinical testing, as a result of perceived, potential or actual liver toxicity issues which outweigh or potentially outweigh potential therapeutic effect of such potential drugs. By co-administering the compositions described herein with such prospective drugs, one may mitigate liver toxicity issues and potentially take advantage of the benefits such drugs may otherwise offer.

An effective amount of the compositions described herein, may typically comprise that amount of such composition that yields a desired treatment effect, e.g., reduction in symptoms of disease, change in levels of biomarkers correlated with a disease, delayed onset of signs or symptoms of a disease in a subject at a heightened risk of such disease, higher tolerance for hepatotoxic drugs or substances, etc. As will be appreciated, the effective amount will vary depending upon the nature of the disorder being treated, the desired extent of an effect, as well as characteristics of the patient, e.g., height, weight, etc.

Treatment of liver disorders may focus on reduction or improvement of one or more symptoms of the disorder in question. In some cases, these symptoms may be physical manifestations of a disorder, e.g., jaundice, fibrosis progression, hypertriglyceridemia, and ascities. In such cases, treatment may include administering the compositions described herein in amounts effective to reduce such physically manifested symptoms, e.g., reduction of jaundice, or in the slowed progression of such symptoms, e.g., slowed fibrosis progression, relative to an untreated subject in a similar situation.

In some cases, the treatment may result in a favorable change in one or more indicators associated or correlated with progression of liver disease, including changes in biomarkers or indicators associated with the condition. A number of diagnostic indicators have been utilized in identifying and characterizing the onset and progression of liver disorders (see, e.g., Chalassani, et al., Hepatology Vol. 67, No. 1 (2018) 328-357). For example, commonly used non-invasive tools for assessing liver disorders include the NAFLD fibrosis score (“NFS”), the FIB-4 index, the aspartate aminotransferase (“AST”) platelet ratio index (“APRI”), and other serum biomarkers, such as enhanced liver fibrosis (“ELF”) panels, as well as imaging techniques, including transient elastography (“TE”) and magnetic resonance (“MR”) elastography, and ultrasonic methods, such as acoustic radiation force impulse imaging and supersonic shear wave elastography.

A number of these diagnostic tools rely upon a range of subject characteristics, including, for example, body mass index, hyperglycemia, and a variety of biomarkers, such as platelet count, aspartate amino transferase (“AST”) and alanine amino transferase (“ALT”) levels or ratios. By way of example, increases in fatty deposits in the liver have been shown to induce inflammatory responses, including secretion of increased levels of transaminase enzymes AST and ALT. As such, AST and ALT are commonly used biomarkers of liver injury associated with hepatotoxicity (Drug Induced Liver Injury—DILI), NAFLD (non-alcoholic fatty liver disease; from NAFL to NASH, fibrosis and cirrhosis), alcoholic hepatitis, and other similar or related liver disorders, alone, together, or as part of an overall scoring and diagnostic tool, as noted above. Liver disease has been identified as a predominant cause of increased transaminase activity in serum. Serum activities of aspartate aminotransferase (AST) and alanine aminotransferase (ALT) have been shown to be elevated when disease processes affect liver cell integrity. Between these two, ALT is more specific enzyme for liver insult, as AST may originate from skeletal and cardiac muscle tissues as well. Alterations of ALT activity persist longer than AST activity. Activities of both enzymes may reach as high as 100-times upper reference limit in liver diseases (See, e.g., Kim W R, Flamm S L, Di Bisceglie A M, et al. Serum activity of alanine aminotransferase (ALT) as an indicator of health and disease. Hepatology. 2008; 47:1363-1370). AST/ALT ratios of greater than 1 have been used as a prediction of cirrhosis, and have shown sensitivity and specificity of 81.3 and 55.3%, respectively. In some etiologies of chronic hepatitis, the ratio may be less than or equal to 1, whereas a ratio of greater than 2 may suggest alcoholic hepatitis (See, e.g., Giannini E, Risso D, Botta F, et al. Validity and clinical utility of the aspartate aminotransferase- alanine aminotransferase ratio in assessing disease severity and prognosis in patients with hepatitis C related chronic liver disease. Arch. Intern. Med. 2003; 163:218-224).

In some cases, the methods of treatment described herein may result in a reduction or other improvement of one or more of the above-described indicators, signs or symptoms of liver injury, including, for example, composite diagnostic scores, e.g,. Fib-4 and/or NFS, AFRI, ELF panels, and the like, as well as imaging and/or acoustic assessment tools, such as TE, MR and ultrasonic methods. Such reductions may comprise and/or result from reductions of one or more of the input parameters for these diagnostic tools, such as liver enzymes (ALT and/or AST) or matrix turnover proteins (hyaluronic acid, tissue inhibitor of metalloproteinase 1 and N-terminal percollagen III peptide, BMI, hyperglycemia metrics, platelet count, or one or more imaging or elastography measures.

As an example, ALT, AST, and AST:ALT ratios, have all been used as indicators of liver disease or injury in diagnostic screening. In particular, normal reference values of AST are generally in the range of 8 to 40 IU/L (˜10-40 in males, and ˜9-32 in females) while normal reference ranges of ALT for adults are from 7 to 55 IU/L. By contrast, in some cases in patients suffering from liver disease or liver injury, these levels may be significantly increased, e.g., 2×, 5×, 10×, or even 20× or more than the normal levels. In clinical diagnostic settings, for male patients, levels of ALT above 30, or female levels above 18 are often viewed as being indicative of an increased risk for NASH/NAFLD.

In some cases, subjects to be treated according to the methods described herein may have starting AST levels (prior to treatment) of at least 15 IU/L, at least 20 IU/L, at least 25 IU/L, at least 30 IU/L, at least 35 IU/L, at least 40 IU/L, at least 45 IU/L, at least 50 IU/L, at least 55 IU/L, at least 60 IU/L, at least 65 IU/L, at least 70 IU/L, at least 75 IU/L, at least 80 IU/L, at least 85 IU/L, at least 90 IU/L, at least 95 IU/L, at least 100 IU/L, at least 110 IU/L, at least 120 IU/L, at least 130 IU/L, at least 140 IU/L, at least 150 IU/L, at least 160 IU/L, at least 170 IU/L, at least 180 IU/L, at least 190 IU/L, at least 200 IU/L or more.

In some cases, subjects to be treated according to the methods described herein may have starting ALT levels (prior to treatment) of at least 10 IU/L, at least 15 IU/L, at least 20 IU/L, at least 25 IU/L, at least 30 IU/L, at least 35 IU/L, at least 40 IU/L, at least 45 IU/L, at least 50 IU/L, at least 55 IU/L, at least 60 IU/L, at least 65 IU/L, at least 70 IU/L, at least 75 IU/L, at least 80 IU/L, at least 85 IU/L, at least 90 IU/L, at least 95 IU/L, at least 100 IU/L, at least 110 IU/L, at least 120 IU/L, at least 130 IU/L, at least 140 IU/L, at least 150 IU/L, at least 160 IU/L, at least 170 IU/L, at least 180 IU/L, at least 190 IU/L, at least 200 IU/L or more.

In some cases, administration of an effective amount of the compositions described herein will reduce symptoms of liver disorders, or delay their onset as described above, and/or will result in a favorable change in the levels of biomarkers associated or correlated with liver disease or the progression of same. In some cases, the above described treatments are administered in effective amounts to reduce AST and/or ALT levels in subjects suffering from liver disease or injury or those at risk of suffering from such disease or injury, e.g., in those subjects demonstrating elevated AST and/or ALT levels. In particular, in some cases, treatment using the above described compositions may yield a reduction in one or both of AST and/or ALT levels in a subject by at least 5 IU/L, in some cases, by at least 10 IU/L, in some cases, by at least 20 IU/L, in some cases by at least 40 IU/L, in some cases by at least 50 IU/L in some cases by at least 60 IU/L in some cases by at least 70 IU/L, in some cases by at least 80 IU/L, in some cases at least 90 IU/L, and in some cases, at least 100 IU/L, 200 IU/L, 300 IU/L, 400 IU/L, 500 IU/L or more.

In some cases, subjects to be treated will have starting AST/ALT ratios that are in excess of 1, in excess of 1.1, in excess of 1.2, in excess of 1.3, in excess of 1.4, in excess of 1.5, in excess of 2, in excess of 5, in excess of 10, in excess of 20, in excess of 30, in excess of 40, in excess of 50, in excess of 60, in excess of 70, in excess of 80, in excess of 90, in excess of 100 or more. In some cases, Following treatment as set forth elsewhere herein, these ratios may be reduced to ratios that approach 1, or are closer to 1 than the starting ratio, including reductions by at least 10%, at least 20%, at least 30%, at least 40%, at least 50%, and in the case of sufficiently high starting ratios, at least 60%, at least 70%, at least 80%, at least 90%, or more.

Effective amounts of the microbial compositions may vary depending upon severity if disorders, the height and weight of the subject, and the relative condition of the subject's gut microbiome, e.g., whether the objective is to over represent the microbial populations in the gut, or to supplement the microbial populations in a deficiently populated gut. In any event, effective amounts of the microbial compositions to be administered for the treatments described herein, with respect to any individual microbial populations, may be described in terms of viable microbes administered to a subject in terms of “colony forming units” or “CFUs”.

In some cases, an effective amount may include administering one or more viable microbial populations to a subject in an aggregate amount of viable microbial populations that is between about 1×10⁷ and 1×10¹⁵ CFUs per administration. In some cases, the administration will be at least 1×10⁷ CFUs of the microbes per administration, at least 1×10⁸ CFUs per administration, at least 1×10⁹ CFUs per administration, at least 1×10¹⁰ CFUs per administration, at least 1×10¹¹ CFUs per administration, at least 1×10¹² CFUs per administration, at least 1×10¹³ CFUs per administration, at least 1×10¹⁴ CFUs per administration or more.

Where multiple microbial populations are present within the composition, each population would make up any fraction of the above aggregate microbial loads. In particular, each microbial population may be present anywhere from 1% or less to 99% or more of the microbial populations present in the composition, or any integer therebetween. The fraction can be calculated based on the number of CFUs of each microbial population. In some cases, any one microbial population may be present within the composition or dose at a level of from about 1×10⁷ and 1×10¹⁵ CFUs per administration, at least 1×10⁷ CFUs of the microbes per administration, at least 1×10⁸ CFUs per administration, at least 1×10⁹ CFUs per administration, at least 1×10¹⁰ CFUs per administration, at least 1×10¹¹ CFUs per administration, at least 1×10¹² CFUs per administration, at least 1×10¹³ CFUs per administration, at least 1×10¹⁴ CFUs per administration or more.

In some cases, the above amounts of viable microbes may be given to a subject once per week, twice per week, three times per week, every other day, 4 times per week, five times per week, six times per week, daily, twice daily, three times daily, four times daily or more.

These administrations may be continued over the course of one day, two days, one week, two weeks, four weeks, six weeks, eight weeks, twelve weeks, eighteen weeks, twenty-six weeks, 7 months, 8 months, 9 months, 10 months, 11 months, one year, between one and two years, two years, between two and three years, three years, between three and four years or more.

In some cases, an effective amount may be administered in a single dose or in multiple doses, and/or in a single administration, or in multiple administrations given over time. An individual dose may be included in an individual administrable form, e.g., a single pill, tablet, chewable, sachet, bar, suppository, or the like, or it may be included in 2, 3, 4, 5, 6, 7, 8, 9, 10 or more individual administrable forms.

Individual doses of the microbial compositions may include, as to any one of the one or more microbial populations included in a given dose between about 1×10⁷ and 1×10¹⁵ CFUs per dose. In some cases, the administration will be at least 1×10⁷ CFUs of the microbes per dose, at least 1×10⁸ CFUs per dose, at least 1×10⁹ CFUs per dose, at least 1×10¹⁰ CFUs per dose, at least 1×10¹¹ CFUs per dose, at least 1×10¹² CFUs per dose, at least 1×10¹³ CFUs per dose, at least 1×10¹⁴ CFUs per dose, or mores. By way of example, for a composition comprising multiple different microbial strains, i.e., 2 or more distinct microbial populations, each population may be present in a single dose at an appropriate fraction of the above described viable microbe load per dose.

In some cases, different microbial populations may be represented to a greater extent than others within a composition, dose or administration. For example, in some cases, one may wish to provide larger proportions of primary and secondary fermenter organisms within the composition and relatively smaller populations of mucin degrading microbes. In other cases, the converse may be desirable. Accordingly, in the case of a consortium of microbial populations present in a given composition, any one microbial population within that consortium of populations may make up anywhere from 1% to 99% of the total microbial load of the composition, and in some cases, will make up 5% or less, from 5% to 10%, up to and including 15%, up to and including 20%, up to and including 25%, up to and including 30%, up to and including 35%, up to and including 40%, up to and including 45%, up to and including 50%, up to and including 55%, up to and including 60%, up to and including 65%, up to and including 70%, up to and including 75%, up to and including 80%, up to and including 85%, up to and including 90%, or up to and including 95% or more of the aggregate microbial load in the composition.

In some cases, treatment of liver disorders may comprise administration of multiple doses over a period of time. In some cases, administration may comprise administration of 1, 2, 3, 4, 5, 6 or more doses over the period of a day. In some cases, such daily administration may occur 1 day, 2 days, 3 days, 4 days, 5 days, 6 days or 7 days during a week. In some cases, such weekly administration may occur over the course of 1 week, 2 weeks, 3 weeks, 4 weeks, 6 weeks, 10 weeks, 12 weeks or longer. In some cases, such longer term administration may occur over the course of 1 month, 2 months, 3 months, 4 months, 5, months, 6 months, 7 months, 8 months, 9 months, 10 months, 11 months, 12 months or longer. In some cases administration may be ongoing in order to maintain effects of such treatment, e.g., with the above described administration occurring over the course of 1 year, 2 years, 3 years, 4 years, 5 years, 6 years, 7 years, 8 years, 9 years, 10 years or longer.

In many cases, administration of the compositions described herein will be by oral/enteral administration. In such cases, the compositions may be formulated in any of a variety of orally ingestible composition types, including, for example, as a capsule, tablet, suspension or emulsion, or as a food like product, such as a chewable, gummy, bar, wafer, cracker, or other edible format that includes the microbial compositions described here. In some cases, the compositions may be administered by other means, including, for example, as suppositories, enemas, or implants administered directly into the gut, e.g., through colonic administration.

In some cases, where formulated for oral administration, the microbial compositions may be contained within an acid resistant matrix, such as an enteric coating, capsule or microcapsule, in order to ensure that maximally viable microbes are able to survive acidic conditions of the stomach, and reach the gut, e.g., the ileum, cecum, etc. A variety of acid resistant materials are available for use in delivering therapeutic and/or biologically active ingredients through the stomach, including for example, hydroxypropyl methyl cellulose (HPMC) and HPMC phthalate encapsulating materials. These materials are generally commercially available as coatings for tablets or as matrices for microencapsulation, or as prefabricated capsules in which the microbial compositions may be packed. In some cases, the capsules and/or coatings, as well as the excipients and other adjunct materials will be free of animal derived products, such as milk, milk proteins, animal derived gelatin, or other animal derived proteins.

In some cases, administration of the compositions described herein may accompany a meal, may precede a meal, or may follow a meal, in order to provide optimal conditions for one or more of transitioning the composition through the stomach into the gut.

Microbial compositions as disclosed herein can be formulated as a supplement, for example, a dietary supplement (e.g., nutritional supplement), or a daily supplement. A dietary supplement can be a product that is taken by mouth that contain a dietary ingredient used to supplement the diet. A dietary supplement can be intended to provide nutrients that may otherwise not be consumed in sufficient quantities; for example, vitamins, minerals, proteins, amino acids or other nutritional substances. In some embodiments, a dietary supplement is not intended to treat, diagnose, cure, or alleviate the effects of a disease or condition. A dietary supplement can be in any form disclosed herein.

Microbial compositions as disclosed herein can be formulated as a medical food. Microbial compositions as disclosed herein can be labeled as a medical food. A medical food can be a food which is formulated to be consumed or administered enterally under the supervision of a physician and which is intended for the specific dietary management of a disease or condition (e.g., a disease or condition disclosed herein), for which distinctive nutritional requirements, based on recognized scientific principles, are established by medical evaluation. In some embodiments, medical foods can be distinguished from the broader category of foods for special dietary use, for example, by the requirement that medical foods are intended to meet distinctive nutritional requirements of a disease or condition, are intended to be used under medical supervision, and are intended for the specific dietary management of a disease or condition. The supervision of a physician can refer to ongoing medical supervision (e.g., in a health care facility or as an outpatient) by a physician who has determined that the medical food is necessary to the subject's overall medical care. The subject can generally see the physician on a recurring basis for, among other things, instructions on the use of the medical food as part of the dietary management of a given disease or condition.

In some embodiments, medical foods are not those simply recommended by a physician as part of an overall diet to manage the symptoms or reduce the risk of a disease or condition. Rather, in some embodiments, medical foods can be foods that are specially formulated and processed (as opposed to a naturally occurring foodstuff used in a natural state) for a subject who requires use of the product, for example, as a major component of a disease or condition's specific dietary management. In some embodiments, medical foods are not regulated as drugs, and do not require a prescription. A medical food can be in any form disclosed herein.

In some embodiments, a composition of the disclosure is a medical food that is used only under medical supervision. In some embodiments, a medical food of the disclosure is used to manage a liver disorder as disclosed herein.

### III. Examples

A viable consortium of microbial species, including both primary and secondary fermenters, was formulated into a dry powder and incorporated into delayed release capsule chosen to release its contents after it exited the stomach of a subject following ingestion.

A double blind, placebo controlled clinical trial employed 23 to 37 patients in each of two test arms and 26 patients in a placebo arm. The two test arms received one of two encapsulated formulations containing different subsets of microbial strains in combination with a prebiotic fiber source and an excipient, while the placebo group received encapsulated formulations including only the prebiotic fiber source and the excipient. The first test arm was administered daily doses of test formulation WBF-010, which included three different microbial strains, two secondary fermenters, Clostridium butyricum (at 3.3×10⁹ CFUs per day), and Clostridium beijerinckii (1.2×10¹⁰ CFUs per day), and a primary fermenter, Bifidobacterium infantis (at 2×10⁹ CFUs per day)

The second test arm additionally included a mucin degrading microbe, Akkermansia muciniphila, at 1.2×10⁹ CFUs per day and an additional secondary fermenter, Eubacterium hallii at 0.9×10⁹ CFUs per day. Both test formulations also included a quantity of prebiotic fiber, and made up the remaining mass with an inert excipient. The placebo arm was administered capsules of the same mass and color, as well as the prebiotic fiber, but with inert excipient substituting for the microbial strain powders.

Subjects were administered 6 capsules per day (3 in the morning and 3 in the evening) over the course of 12 weeks, and were given blood tests at 0, 2 and 4 weeks after commencing treatment, and at the 12 week date to test for ALT and AST, among other markers relevant to metabolic disorders being tested.

FIG. 2 shows plots of AST and ALT levels, and changes in AST and ALT levels from time 0, in each of the patient arms (placebo, WB-010 and WB-011). As shown, those subjects receiving the placebo showed an increase in both ALT and AST levels from their initial levels to those levels at the completion of the study. Conversely, those subjects receiving one of the test compositions that included the microbial consortia, showed lower levels of both AST and ALT levels, with those subjects receiving the WBF-010 formulation showing a modest decrease in levels vs. their starting point, and the WBF-011 formulation demonstrating the greatest reduction over time and a clinically significant reduction over the placebo group.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A composition for use in treating a subject suffering or at risk of developing a liver disorder, the composition comprising a microbial population.

2. The composition for use of claim 1, wherein the composition further comprises at least one of a preservative and an enteric coating.

3. The composition for use of any of claim 1 and claim 2, wherein the composition further comprises fiber.

4. The composition for use of any of claims 1-3, wherein the microbial population comprises an rRNA sequence comprising at least 85% sequence identity to an rRNA sequence of Akkermansia muciniphila, Bifidobacterium adolescentis, Bifidobacterium infantis, Bifidobacterium longum, Clostridium beijerinckii, Clostridium butyricum, Clostridium indolis, or Eubacterium hallii.

5. The composition for use of any of claims 1-4, wherein the microbial population is a viable population.

6. The composition for use of any of claims 1-5, wherein the composition comprises at least one primary fermenter and at least one secondary fermenter.

7. The composition for use of any of claims 1-6, wherein the microbial population has a viability of at least 1×10\hat{ }5 CFU/g of the composition.

8. The composition for use of any of claims 1-7, wherein the composition is dairy-free.

9. The composition for use of any of claims 1-8, wherein the composition is a pill, capsule, tablet, gummy, or a chewable tablet.

10. The composition for use according to any of claims 1-9, wherein the composition, when administered to the subject, reduces a serum level of aspartate transaminase (AST) or alanyl transaminase (ALT) by at least 5 IU/L in the subject as compared to ALT and/or AST levels in the subject prior to administering the microbial population.

11. A method of treating a subject suffering from or at risk of developing a liver disorder, the method comprising:
administering to the subject an effective amount of a microbial composition and
reducing a serum level of aspartate transaminase (AST) or alanyl transaminase (ALT) by at least 5 IU/L in the subject as compared to ALT and/or AST levels in the subject prior to administering the microbial composition.

12. The method according to claim 11, wherein the liver disorder is selected from the group consisting of nonalcoholic steatohepatitis (NASH), non-alcoholic fatty liver disease (NAFLD), liver fibrosis, cirrhosis, alcohol induced liver disease, and drug induced liver injury.

13. The method according to any of claims 11-12, wherein the liver disorder is selected from the group consisting of: NASH and NAFLD.

14. The method according to any of claims 11-12, wherein the subject suffers from a metabolic disorder.

15. The method according to claim 14, wherein the metabolic disorder is selected from the group consisting of type 1 diabetes mellitus, type 2 diabetes mellitus, insulin resistance, and obesity.

16. The method according to any of claims 10-15, wherein the microbial composition is formulated in an ingestible form and is administered orally.

17. The method according to claim 16, wherein the ingestible form comprises a powder.

18. The method according to any of claims 16-17, wherein the microbial composition comprises microbes that are microencapsulated in the ingestible form.

19. The method according to any of claims 11-18, wherein the microbial composition comprises 2 or more microbial species selected from primary fermenters and secondary fermenters.

20. The method according to any of claims 11-19, wherein the microbial composition comprises 2 or more microbial species selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof.

21. The method according to any of claims 11-20, wherein the microbial composition comprises 2 or more microbial species selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii.

22. The method of any of claims 11-21, wherein the administration reduces the serum level of one or more of aspartate transaminase (AST) and alanyl transaminase (ALT) enzymes by at least 10 IU/L in the subject as compared to ALT and/or AST levels in the subject prior to administering the microbial composition.

23. The method according to any of claims 11-22, wherein the administration reduces the serum level of one or more of aspartate transaminase (AST) and alanyl transaminase (ALT) enzymes by at least 20 IU/L in the subject as compared to ALT and/or AST levels in the subject prior to administering the microbial composition.

24. The method according to any of claims 11-23, wherein the administration reduces the serum level of one or more of aspartate transaminase (AST) and alanyl transaminase (ALT) enzymes by at least 50 IU/L in the subject as compared to ALT and/or AST levels in the subject prior to administering the microbial composition.

25. The method according to any of claims 11-24, wherein the administration reduces the serum level of one or more of aspartate transaminase (AST) and alanyl transaminase (ALT) enzymes by at least 100 IU/L in the subject as compared to ALT and/or AST levels in the subject prior to administering the microbial composition.

26. A method of reducing one or more elevated indicators of liver injury or disease in a subject, comprising administering to a subject having one or more elevated indicators of liver injury an effective amount of a composition comprising a viable microbial population, wherein the microbial population is capable of producing butyrate in a gut of the subject, thereby reducing at least one of the one or more indicators of liver disease in the subject.

27. The method according to claim 26, wherein the one or more indicators of liver injury are selected from the group consisting of: AST, ALT, AST:ALT ratio, fibrosis score (“NFS”), the FIB-4 index, the aspartate aminotransferase (“AST”) platelet ratio index (“APRI”), enhanced liver fibrosis (“ELF”) panels, transient elastography (“TE”), magnetic resonance (“MR”) elastography, acoustic radiation force impulse imaging, and supersonic shear wave elastography.

28. A method of lowering one or more of serum ALT and AST levels in a subject suffering from a liver disorder or at risk of suffering from a liver disorder, the method comprising:
administering to the subject an effective amount of a consortium of isolated and purified microbial species to lower the one or more serum ALT and AST levels in the subject.

29. The method according to claim 28, wherein the subject suffers from type 2 diabetes.

30. The method according to claim 29, wherein the subject has been diagnosed with a liver disorder.

31. The method according to claim 30, wherein liver disorder is NAFLD, NASH, liver fibrosis, cirrhosis, or DILI.

32. The method according to claim 31, wherein the subject is concurrently administered a drug with known liver toxicity.

33. A method of reducing the risk of developing liver toxicity associated with one or more drug compounds known to have liver toxicity in a subject, the method comprising:
administering to the subject the one or more drug compounds, and
administering to the subject a composition comprising a microbial population in an amount effective to lower one or more indicators of liver injury.

34. A method of treating a liver disorder in a subject in need thereof, comprising:
administering to the subject an effective amount of a consortium of isolated and purified microbial species to mitigate the liver disorder.
